(19) United States
(12) Patent Application Publication
(10) Pub. No.: US 2024/0099813 A1
(43) Pub. Date: Mar. 28, 2024

(54) ORTHODONTIC PLAN DISPLAY SYSTEM

(71) Applicant: MEDIT CORP., Seoul (KR)

(72) Inventors: Sunghoon LEE, Seoul (KR); Jinyoung KIM, Seoul (KR)

(73) Assignee: MEDIT CORP., Seoul (KR)

(21) Appl. No.: 18/274,627

(22) PCT Filed: Jan. 20, 2022
(86) PCT No.: PCT/KR2022/001026

§ 371 Date: Jul. 27, 2023

(30) Foreign Application Priority Data
Jan. 28, 2021 (KR) 10-2021-0012266
Jul. 23, 2021 (KR) 10-2021-0097153

(51) Int. Cl. A61C 7/00; A61C 9/00; G16H 20/40; G16H 50/50

(52) U.S. Cl. CPC A61C 7/002; A61C 9/0046; G16H 20/40; G16H 50/50; A61C 2007/004

(57) ABSTRACT

The present disclosure relates to an orthodontic plan display system and, more particularly, to an orthodontic plan display system for displaying changes in the position of teeth which are to receive orthodontic treatment, by using oral scan data obtained by scanning a shape of an oral cavity of a patient for orthodontic treatment. The orthodontic plan display system of the present invention has an effect of being conveniently used for orthodontic treatment by establishing an orthodontic plan for a process of fixing some teeth for orthodontic treatment of a patient and carrying out orthodontic treatment only for the remaining teeth, thereby making it possible to visually check the process.

### TECHNICAL FIELD

The present disclosure relates to an orthodontic plan display system and, more particularly, to an orthodontic plan display system for displaying changes in the position of teeth subject to orthodontic treatment using oral scan data obtained by scanning the shape of the oral cavity of a patient for orthodontic treatment.

### BACKGROUND

It is important to clearly identify the oral structure of a patient and decide which teeth to move in which direction and by how much for correction during orthodontic treatment. Recently, a system capable of simulating a process of scanning the shape of an oral cavity using an oral scanner to individualize each tooth and correcting teeth into an ideal oral structure by orthodontic algorithm has been developed and used.

In the case of a conventional orthodontic simulation system, it is common to simulate an orthodontic state by adjusting the positions and directions of all teeth in the mouth.

However, in the case of actual orthodontic treatment, there are frequent cases in which orthodontic treatment is performed only on some teeth depending on the patient's oral condition. That is, orthodontic treatment may be performed only on incisors, or orthodontic treatment may be performed only on molars. In addition, in some cases, orthodontic treatment may be performed only on some teeth, and then orthodontic treatment may be further performed on other teeth in stages.

As described above, instead of performing orthodontic treatment on all teeth, if the process of performing orthodontic treatment only on the remaining teeth while some teeth are fixed is effectively simulated and visualized, it may be conveniently used in establishing an orthodontic plan. In addition, the orthodontic plan display system may be conveniently used by orthodontists to explain the stepwise process of orthodontic treatment to patients to facilitate their understanding.

### SUMMARY

The present disclosure was made to meet the above-mentioned needs and aims to provide an orthodontic plan display system capable of easily and visually simulating the process of fixing some teeth and performing orthodontic treatment only on the remaining teeth using the shape of an oral cavity, which was obtained from oral scan data.

In view of the foregoing, an orthodontic plan display system according to the present disclosure may include: an oral information storage module configured to store oral information including oral scan data obtained by scanning the shape of an oral cavity and tooth area data for dividing respective tooth areas among the oral scan data; a fix information receiving module configured to receive, from an input device, a selection of at least one tooth, which is not to be corrected and whose position is to be fixed from among teeth of the oral information, and receive the tooth as fix information; an automatic arrangement module configured to generate orthodontic information for correction in which the positions and directions of the remaining teeth, excluding the teeth fixed by the fix information among the teeth of the oral information, are automatically arranged according to an orthodontic algorithm; and an oral display module configured to display a corrected oral shape according to the orthodontic information generated by the automatic arrangement module on a display device.

The orthodontic plan display system of the present disclosure has an effect of being conveniently used for orthodontic treatment by establishing an orthodontic plan for a process of fixing some teeth of a patient for orthodontic treatment and performing orthodontic treatment only on the remaining teeth, and making it possible to visually check the process.

### BRIEF DESCRIPTION OF DRAWINGS

FIG. 1 is a block diagram of an orthodontic plan display system according to an embodiment of the present disclosure.

FIGS. 2 to 7 illustrate screens displayed on a display device to explain the operation of the orthodontic plan display system shown in FIG. 1.

### DETAILED DESCRIPTION

Hereinafter, an orthodontic plan display system according to an embodiment of the present disclosure will be described with reference to the accompanying drawings.

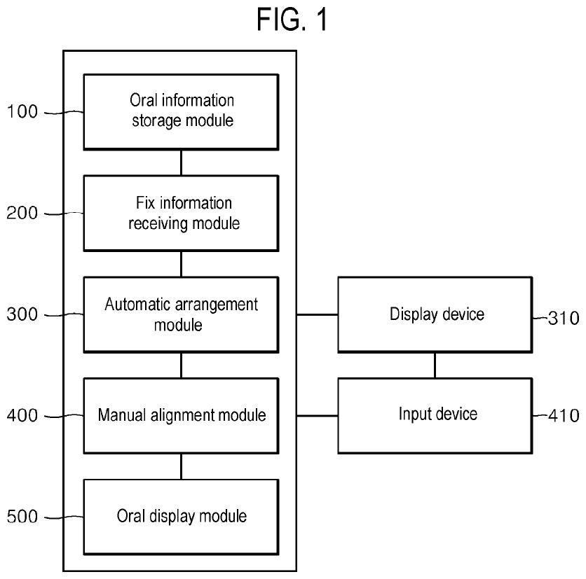

FIG. 1 is a block diagram of an orthodontic plan display system according to an embodiment of the present disclosure.

The orthodontic plan display system of the present disclosure is intended to visually identify an oral structure during or after orthodontic treatment of a patient on the basis of the structure and shape of the patient's teeth obtained by scanning the oral structure.

Referring to FIG. 1, the orthodontic plan display system of the present embodiment includes an oral information storage module 100, a fix information receiving module 200, an automatic arrangement module 300, a manual alignment module 400, and an oral display module 500.

Oral information is stored in the oral information storage module 100. The oral information includes oral scan data and teeth area data. The oral information further includes tooth numbers of individual teeth. The oral information may also include tooth numbers of missing teeth as additional information.

In the case of the present embodiment, oral scan data obtained by three-dimensionally scanning the oral structure of a patient is received and stored in the oral information storage module 100. It is possible to identify the area corresponding to the teeth in the shape of the oral cavity by analyzing the above oral scan data. In addition, portions corresponding to the teeth area may be separated into shape information for each tooth, and a tooth number may be assigned to such an individual tooth.

As described above, the process of separating the teeth area from the oral scan data and the process of automatically assigning the tooth numbers to the respective teeth may be performed in a separate device, and organized oral information thereof may be stored in the oral information storage module 100. Alternatively, the orthodontic plan display system of the present disclosure may receive an input of oral scan data and extract oral information therefrom, thereby storing the oral information in the oral information storage module 100.

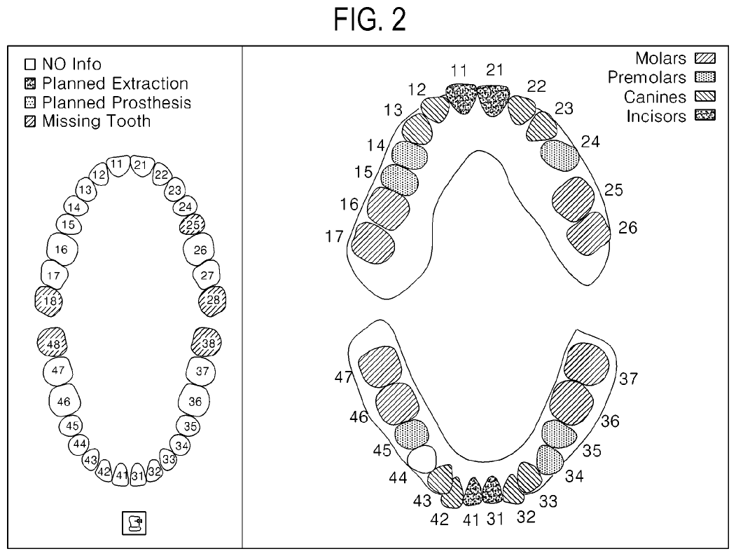

The oral display module 500, as described above, displays the shape of the oral cavity and the tooth numbers of the respective teeth stored in the oral information storage module 100 as the oral information on the display device 310. FIG. 2 shows an example in which the shape of the oral cavity and respective tooth numbers according to the oral information are displayed on the display device 310. In FIG. 2, the left screen of the display device 310 shows a standard teeth model displaying tooth numbers and missing teeth, and the right side thereof shows the shape of the oral cavity and the tooth numbers of the respective teeth according to the oral information.

The fix information receiving module 200 plays a role of designating teeth that are to be fixed in position instead of being treated, in performing orthodontic treatment simulation. The oral display module 500 displays, on the screen, an input tool for designating teeth to be fixed among the teeth of the teeth model displayed on the display device 310. The fix information receiving module 200 receives a selection of teeth to be fixed through an input device 410 such as a mouse, and receives and stores tooth numbers thereof as fix information.

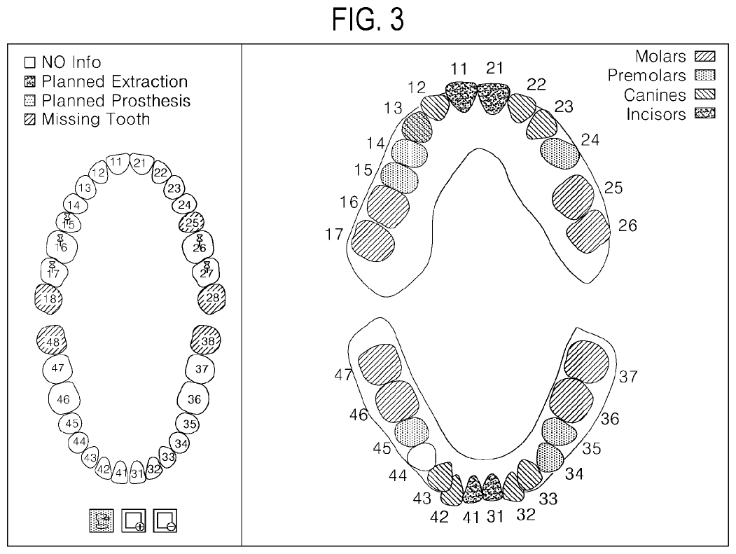

In the case of the teeth model shown in FIG. 3, teeth 15, 16, 17, 26, and 27 are respectively designated as teeth to be fixed by clicking on the same with a mouse. The display device 310 displays the teeth to be fixed as “tacks” on the standard teeth model so that the user may easily identify the teeth to be fixed.

As described above, the fix information receiving module 200 receives, through the input device 410, the tooth numbers by selecting at least one of the teeth in the oral information displayed on the display device 310. Various tools of a computer system, such as a mouse, a touch pad, a touch screen, a keyboard, and a track pad, may be used as the input device 410 to receive the fix information.

The automatic arrangement module 300 automatically arranges the teeth of the oral information according to an orthodontic algorithm to generate orthodontic information enabling a desirable oral shape. At this time, the automatic arrangement module 300 maintains the state of the teeth fixed according to the fix information received by the fix information receiving module 200 and automatically arranges the positions and directions of the remaining teeth to generate orthodontic information. Various well-known methods may be used as an algorithm for generating the orthodontic information by automatically arranging the teeth by the automatic arrangement module 300. In general, the automatic arrangement module 300 generates the orthodontic information such that an arch line is extracted from the shape of the oral cavity according to the oral information and such that the teeth are aligned and arranged along the arch line.

The automatic arrangement module 300 may use two methods in automatically arranging the remaining teeth while fixing the teeth to be fixed according to the fix information.

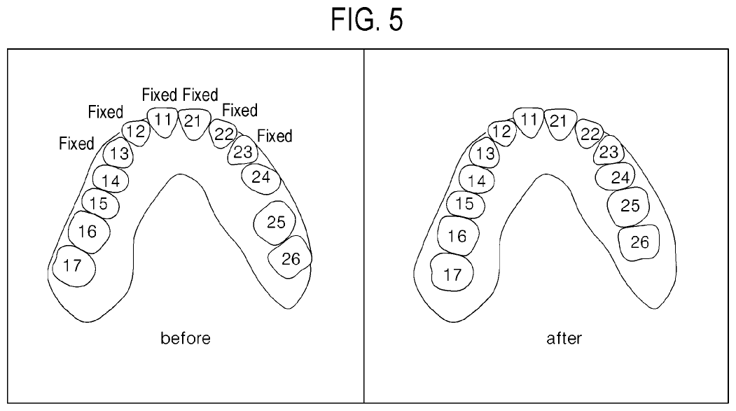

First, the automatic arrangement module 300 may automatically arrange the teeth such that the remaining teeth sequentially come into contact with each other based on the teeth to be fixed. In this case, as shown in FIG. 5, since adjacent teeth are aligned in contact with each other, spacing or overlapping between the teeth does not occur.

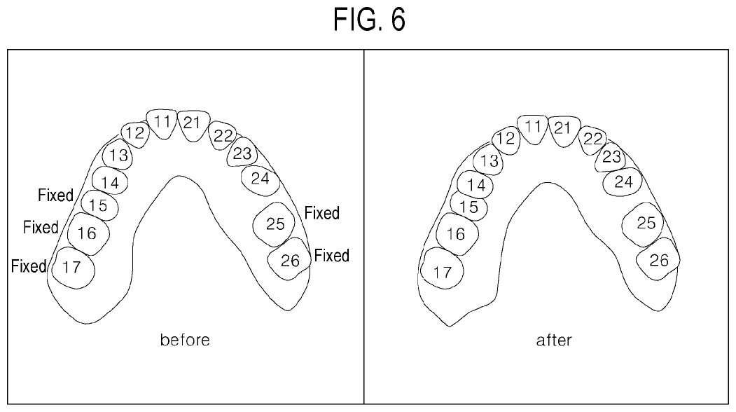

Second, the automatic arrangement module 300 may automatically arrange the teeth such that only the remaining teeth come into contact with each other without considering the teeth to be fixed. That is, only the remaining teeth, excluding the teeth to be fixed, are arranged in appropriate positions and directions according to the orthodontic algorithm. In this case, as shown in FIG. 6, overlap may occur between the teeth to be fixed and the remaining teeth, or gaps may occur between the teeth because they do not contact each other.

The automatic arrangement module 300 may generate orthodontic information by a combination of the above two methods as necessary.

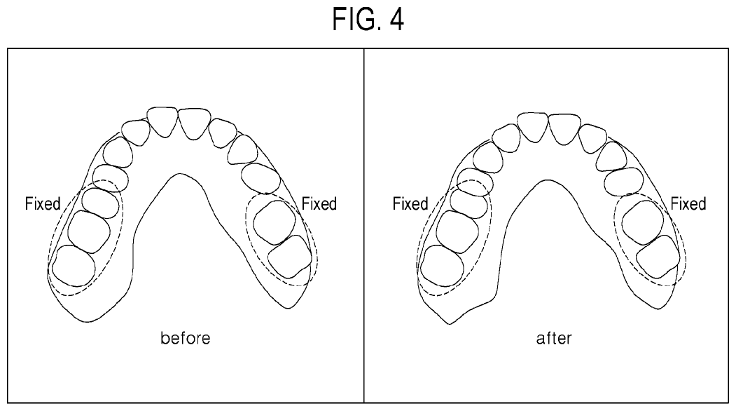

As shown in FIGS. 4 to 6, the oral display module 500 respectively displays the shape of the oral cavity according to the oral information and the corrected shape of the oral cavity according to the orthodontic information on the display device 310. The oral display module 500 may display one of either the oral shape before correction or the oral shape after correction according to the user's selection. Alternatively, the oral display module 500 may simultaneously display the oral shapes before and after correction on the display device 310 as shown in FIGS. 4 to 6.

The manual alignment module 400 receives manual alignment information for adjusting the positions of the teeth fixed by the fix information according to the positions of the teeth automatically arranged by the automatic arrangement module 300, and adjusts the positions of the teeth fixed by the fix information according to the manual alignment information. The oral display module 500 displays a tool capable of receiving manual alignment information on the screen of the display device 310. The user may adjust the positions of the teeth to be fixed using the input device 410 such as a mouse. The manual alignment module 400 receives the adjusted positions of the teeth to be fixed and generates manual alignment information. The oral display module 500 displays the corrected shape of the oral cavity, which was adjusted according to the manual arrangement information, on the display device 310.

In some cases, the manual alignment module 400 may receive manual alignment information for completely realigning the teeth fixed by the fix information. In this case, the manual alignment module 400 generates manual alignment information for realigning the teeth to be fixed such that the teeth to be fixed sequentially come into contact with each other (that is, so as not to be spaced apart from or overlap each other) on the basis of the nearest teeth among the teeth automatically arranged by the automatic arrangement module 300.

At this time, the manual alignment module 400, unlike the method in which the automatic arrangement module 300 automatically arranges the teeth to be corrected, realigns only the positions of the teeth to be fixed without changing the directions thereof That is, the manual alignment module 400 performs only the change and realignment of the positions for moving the teeth to be fixed to the mesial or distal side or moving the same to the lingual or buccal side.

In addition, the manual alignment module 400 may further include information about extracting some of the teeth fixed by the fix information as manual alignment information and adjust and arrange the positions of the remaining fixed teeth, excluding the teeth to be extracted. That is, the manual alignment module 400 is able to simulate a correction process of extracting some of the teeth to be fixed and securing enough space for the remaining teeth to be corrected.

Hereinafter, the operation of the orthodontic plan display system configured as described above will be described.

First, the oral display module 500 displays the shape of the oral cavity on the screen of the display device 310 using oral information stored in the oral information storage module 100.

As shown in FIG. 2, the oral display module 500 displays the shape of the oral cavity to be corrected on the display device 310. In some cases, the tooth numbers may be further displayed on the respective teeth, and other oral information such as whether or not a tooth is subject to extraction or classification of teeth such as molars, premolars, and incisors may be further displayed.

In this state, if the user manipulates a mouse (input device 410) to place a mouse cursor on the teeth to be fixed and clicks, the oral display module 500, as shown in FIG. 3, displays a menu capable of designating the teeth selected by the mouse as teeth to be fixed. FIG. 3 illustrates a state in which teeth 15, 16, 17, 26, and 27 are respectively designated as teeth to be fixed. The oral display module 500 shows the teeth to be fixed by displaying tacks on the corresponding tooth numbers of the standard teeth model so that the user can easily identify the teeth to be fixed. For reference, in the teeth model shown in FIG. 3, teeth 18, 25, and 28 of the upper jaw correspond to missing teeth.

The fix information receiving module 200 receives and stores, as fix information, the numbers of the teeth to be fixed designated by the user as described above.

If the fix information receiving module 200 receives the fix information described above, the automatic arrangement module 300 automatically arranges the remaining teeth according to the orthodontic algorithm while fixing the teeth to be fixed as designated by the fix information to implement the shape of the oral cavity in an ideal orthodontic state and generates orthodontic information according thereto.

At this time, the orthodontic plan display system according to the present embodiment automatically arranges the remaining teeth excluding the teeth to be fixed according to the two options described above. That is, the automatic arrangement module 300 may use a method of automatically arranging the remaining teeth while allowing overlapping and spacing with respect to the teeth to be fixed and a method of automatically arranging the remaining teeth without allowing overlapping and spacing with respect to the teeth to be fixed. One of the two methods may be determined to be used by a user's choice or by the automatic arrangement module 300 depending on the positional relationship between the teeth to be fixed and the remaining teeth.

Hereinafter, a case in which the automatic arrangement module 300 automatically determines the automatic arrangement method according to the positional relationship between the teeth to be fixed and the remaining teeth will be described.

FIG. 5 illustrates a case where incisors are fixed and where teeth behind the same are automatically arranged. FIG. 5 illustrates the states before and after distal teeth 14 to 17 and 24 to 26 are automatically arranged while teeth 11 to 13 and 21 to 23 corresponding to incisors are designated as teeth to be fixed. If the fixed teeth are incisors, the remaining teeth are automatically arranged such that the teeth positioned on the distal side relative to the fixed teeth sequentially come into contact with the teeth fixed according to the fix information. It is orthodontically desirable to arrange the teeth positioned on the distal side with respect to the teeth to be fixed so as to sequentially come into contact with the teeth to be fixed.

FIG. 6 illustrates a case where the molars are fixed and where the front teeth thereof are automatically arranged. FIG. 6 illustrates the states before and after teeth 11 to 14 and 21 to 24 on the mesial side with respect to the teeth to be fixed are automatically arranged while teeth 15 to 17 and 25 to 26 corresponding to molars are designated as teeth to be fixed. If the fixed teeth are molars as described above, the remaining teeth are automatically arranged such that the teeth positioned on the mesial side with respect to the fixed teeth may overlap and may be spaced apart from the teeth fixed by the fix information. That is, the automatic arrangement module 300 arranges the teeth according to the orthodontic algorithm by considering only the positions and states of the remaining teeth on the mesial side without considering the positions of the teeth to be fixed. In this case, as shown in FIG. 6, overlap (between teeth 14 and 15) or separation (between teeth 24 and 25) occurs between adjacent teeth. In the case where the molars are fixed and where the incisors are corrected, there may not be enough space between both molars to correct and arrange the incisors, or the center line of the mouth may be distorted. Therefore, in this case, it is orthodontically desirable to perform automatic alignment considering only the positions and directions of the remaining teeth (incisors) without considering the positions of the fixed molars. The overlap of teeth resulting therefrom may be solved by interproximal reduction (IPR) or tooth extraction, and the gap between the separated teeth may be solved by installing implants.

Although whether or not to allow overlapping or spacing of the remaining teeth with respect to the teeth to be fixed has been described above on the basis of the example in which the teeth to be fixed are incisors and molars with reference to FIGS. 5 and 6, the method in which the automatic arrangement module automatically arranges the remaining teeth is not limited to the above method. The automatic arrangement module may arrange the remaining teeth such that the teeth on the distal side with respect to the teeth to be fixed are arranged to sequentially come into contact with the teeth to be fixed and such that the teeth on the mesial side are arranged to overlap or be spaced apart from the teeth to be fixed.

As described above, the orthodontist is able to effectively establish an orthodontic plan using the orthodontic plan display system of the present disclosure and easily identify the difference in the shape of the oral cavity before and after correction. In addition, the orthodontist is able to explain the orthodontic plan to the patient in a visual and intuitive way using the orthodontic plan display system of the present disclosure. In particular, an orthodontic plan for the remaining teeth may be established in the state of entering the fixation conditions by easily designating the teeth to be fixed, which are not to be corrected, by the fix information module, and the results before and after establishing the plan may be identified, thereby enabling a realistic correction simulation reflecting the process of actual orthodontic treatment.

Meanwhile, as shown in FIG. 6, whether to leave the state in which the teeth to be fixed overlap or are spaced apart from the adjacent teeth as a result of the automatic arrangement of the automatic arrangement module 300 or to align the same in a visually natural state is determined by the user's choice. The overlapping or spacing may remain when used only for a diagnostic or therapeutic orthodontic plan simulation. When the orthodontic plan display system of the present disclosure is used for patient counseling, it may be better to display, on the display device 310, the state in which overlapping or spacing is eliminated for the patient's satisfaction. Therefore, it is desirable to provide an option for the user to select whether or not to eliminate overlapping or spacing.

In the present embodiment, such overlapping or spacing may be solved by the manual alignment module 400.

The manual alignment module 400 receives manual alignment information for adjusting the positions of the teeth fixed by the fix information according to the positions of the teeth automatically arranged by the automatic arrangement module 300 and adjusts the positions of the teeth fixed by the fix information according to the manual alignment information. The user may directly input a command to adjust the positions of the teeth to be fixed using the input device 410 such as a mouse, and the manual alignment module 400 may receive the command and adjust the positions of the teeth to be fixed.

In some cases, the manual alignment module 400 may receive a command for automatically realigning the teeth to be fixed according to the teeth corrected by orthodontic information without overlapping or spacing, instead of receiving specific realignment positions of the teeth to be fixed through the input device 410.

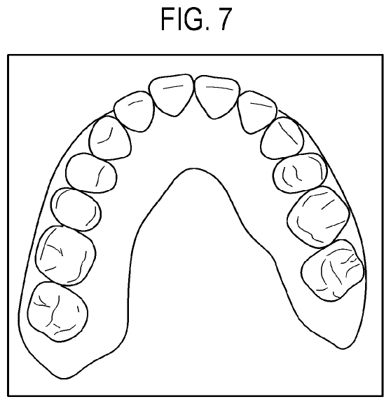

In this case, the manual alignment module 400 adjusts the positions of the teeth to be fixed such that the teeth fixed by the fix information are positioned to come into contact with the closest teeth among the teeth automatically arranged in the state where adjacent teeth are in contact with each other. Accordingly, the teeth to be fixed are realigned to be in contact with adjacent teeth without overlapping other teeth and without being spaced apart from the adjacent teeth. FIG. 7 illustrates a state in which the manual alignment module 400 realigns the automatic alignment result in the state shown in FIG. 6 by eliminating overlapping and spacing.

At this time, the manual alignment module 400 eliminates overlapping and spacing for visual naturalness, instead of automatically aligning the directions of the teeth to be fixed to conform to the arch line for the purpose of correction. Therefore, it is better to adjust and realign only the positions of the teeth fixed by the fix information while the directions thereof are fixed. That is, the manual alignment module 400 performs changes of positions for simply moving the teeth to be fixed to the mesial or distal side, or to the lingual or buccal side, instead of executing complicated calculations according to the orthodontic algorithm, thereby performing only the realignment to the extent that the final result of correction is expressed visually and naturally. By this method, it is possible to display high-quality simulation results by the oral display module 500 while omitting unnecessary calculations.

Meanwhile, the manual alignment module 400 may further include information for extracting some of the teeth fixed by fix information as manual alignment information. In this case, the positions of the remaining fixed teeth, excluding the teeth to be extracted, may be adjusted, thereby performing realignment in the same way as above. The user who has a plan to correct other teeth after tooth extraction without correcting the positions and directions may configure to extract some of the teeth to be fixed by the manual alignment module 400 as described above, thereby effectively identifying a correction simulation result thereof

In addition, as described above, if the teeth corrected by the automatic arrangement module 300 and the teeth to be fixed overlap or are spaced apart from each other, the manual alignment module 400 may calculate overlapping or spacing dimensions between the teeth to be fixed and the teeth corrected by the automatic arrangement module 300. The oral display module 500 displays the overlapping or spacing dimensions calculated by the manual alignment module 400 on the display device 310. The user may correct the orthodontic plan or establish an additional orthodontic plan using the overlapping or spacing dimensions. For example, a value calculated by the manual alignment module 400 may be used as reference data for additional corrections such as IPR, tooth extraction, implant placement, and the like.

Although the preferred examples of the present disclosure have been described above, the scope of the present disclosure is not limited to the examples described and illustrated above.

For example, although the orthodontic plan display system of the above-described embodiment has been described as including the manual alignment module 400, it is also possible to configure an orthodontic plan display system having a structure excluding the manual alignment module 400.

In addition, when the automatic arrangement module 300 generates orthodontic information, whether or not to allow overlapping or spacing of the teeth to be fixed by fix information may be determined by various methods other than the method described above, and a combination of methods of allowing or disallowing overlapping or spacing may vary.

In addition, the method in which the oral display module 500 displays the shape of the oral cavity according to the oral information or the method in which the teeth to be fixed according to the fix information is displayed on the display device 310 may be modified in various ways as needed.

In addition, the algorithm in which the automatic arrangement module 300 corrects and automatically arranges the remaining teeth, excluding the fixed teeth of the fix information, may use various methods as needed.

In addition, although it has been described above that the manual alignment module 400 performs realignment by fixing the directions of the teeth to be fixed and changing only the positions thereof, in some cases, the manual alignment module may be operated to perform realignment to adjust the directions of the teeth to be fixed as well.

1. An orthodontic plan display system comprising:
an oral information storage module configured to store oral information comprising oral scan data obtained by scanning a shape of an oral cavity and tooth area data for dividing respective tooth areas among the oral scan data;
a fix information receiving module configured to receive, from an input device, a selection of at least one tooth, which is not to be corrected and whose position is to be fixed, from among teeth of the oral information to receive the at least one of tooth as fix information;
an automatic arrangement module configured to generate orthodontic information for correction in which positions and directions of the remaining teeth, excluding the at least one tooth fixed by the fix information, among the teeth of the oral information, are automatically arranged according to an orthodontic algorithm; and
an oral display module configured to display a corrected shape of the oral cavity according to the orthodontic information generated by the automatic arrangement module on a display device.

2. The orthodontic plan display system of claim 1, wherein the automatic arrangement module is configured to generate the orthodontic information for automatically arranging the non-fixed remaining teeth to come into contact with the at least one tooth fixed by the fix information.

3. The orthodontic plan display system of claim 1, wherein the automatic arrangement module is configured to generate the orthodontic information for automatically arranging the non-fixed remaining teeth to allow overlapping and spacing with respect to the at least one tooth fixed by the fix information.

4. The orthodontic plan display system of claim 1, wherein the automatic arrangement module is configured to:
generate the orthodontic information for automatically arranging the non-fixed remaining teeth on the distal side of the at least one tooth fixed by the fix information to come into contact with the at least one tooth fixed by the fix information; and
generate the orthodontic information for automatically arranging the non-fixed remaining teeth on the mesial side of the at least one tooth fixed by the fix information to allow overlapping and spacing with respect to the at least one tooth fixed by the fix information.

5. The orthodontic plan display system of claim 1, wherein the automatic arrangement module is configured to:
generate, if the at least one tooth fixed by the fix information is an incisors, the orthodontic information for automatically arranging the remaining non-fixed remaining teeth to come into contact with the at least one tooth fixed by the fix information; and
generate, if the at least one tooth fixed by the fix information is a molars, the orthodontic information for automatically arranging the remaining non-fixed remaining teeth to allow overlapping and spacing with respect to the at least one tooth fixed by the fix information.

6. The orthodontic plan display system of claims 1, further comprising a manual alignment module configured to receive manual alignment information for adjusting the positions of the at least one tooth fixed by the fix information to conform to the positions of the teeth automatically arranged by the automatic arrangement module and adjust the positions of the at least one tooth fixed by the fix information according to the manual alignment information,
wherein the oral display module is configured to display the corrected shape of the oral cavity adjusted according to the manual alignment information generated by the manual alignment module on the display device.

7. The orthodontic plan display system of claim 6, wherein the manual alignment module is configured to adjust, if the manual alignment information for realigning the at least one tooth fixed by the fix information is received, the positions of at least one teeth fixed by the fix information to be positioned such that the at least one teeth fixed by the fix information come into contact with the closest teeth among the teeth automatically arranged in the state of being in contact with adjacent teeth.

8. The orthodontic plan display system of claim 6, wherein the manual alignment module is configured to adjust and realign, if the manual alignment information for realigning the at least one tooth fixed by the fix information is received, the positions of the at least one tooth fixed by the fix information while the directions thereof are fixed.

9. The orthodontic plan display system of claim 6, wherein the manual alignment module further comprises information for extracting some of the at least one tooth fixed by the fix information comprises multiple teeth fixed by the fix information, as the manual alignment information and is configured to adjust and arrange the positions of remaining the multiple teeth fixed by the fix information, excluding the teeth to be extracted.

10. The orthodontic plan display system of claim 6, wherein the manual alignment module is configured to calculate, if overlapping or spacing occurs between at least one tooth fixed by the fix information and teeth for which orthodontic information is generated by the automatic arrangement module, overlapping or spacing dimensions thereof, and
wherein the oral display module is configured to display the overlapping or spacing dimensions of the teeth, which were calculated by the manual alignment module on the display device.

11. The orthodontic plan display system claims 1, wherein the automatic arrangement module is configured to generate the orthodontic information by automatically arranging the remaining teeth, excluding the at least one tooth fixed by the fix information, along an arch line obtained from the oral scan data of the oral information.
